(19) United States
(12) Patent Application Publication
(10) Pub. No.: US 2022/0327735 A1
(43) Pub. Date: Oct. 13, 2022

(54) ULTRASOUND PROBE POSITION REGISTRATION METHOD, ULTRASOUND IMAGING SYSTEM, ULTRASOUND PROBE POSITION REGISTRATION SYSTEM, ULTRASOUND PROBE POSITION REGISTRATION PHANTOM, AND ULTRASOUND PROBE POSITION REGISTRATION PROGRAM

(71) Applicant: FUJIFILM Healthcare Corporation, Kashiwa-shi (JP)

(72) Inventors: Takafumi Shimamoto, Chiba (JP); Nobutaka Abe, Chiba (JP)

(21) Appl. No.: 17/700,735

(22) Filed: Mar. 22, 2022

(30) Foreign Application Priority Data
Apr. 13, 2021 (JP) 2021-067915

(51) Int. Cl. G06T 7/73; G01S 15/89

(52) U.S. Cl. CPC G06T 7/74; G01S 15/8993; G06T 2207/10136; G06T 2207/30204

(57) ABSTRACT

To register a position and an angle of a scanning surface of an ultrasound probe easily and accurately. A phantom including two or more wires stretched in a non-parallel manner is disposed in a real space in which a position detection sensor is disposed. An ultrasound probe, to which a probe position detection marker is attached, is moved on the phantom in a parallel manner while keeping an orientation of a main plane of the ultrasound probe constant. Two or more ultrasound images of the phantom are acquired while detecting a position of the probe position detection marker in the real space with the position detection sensor. Positions of cross-sectional images of the two or more wires included in each of the two or more ultrasound images are obtained. A relation between the position of the probe position detection marker in the real space and orientations and positions of the captured ultrasound images in the real space is calculated based on a relation between the obtained positions of the cross-sectional images. The calculated relation as probe coordinate transformation information is registered in a storage unit.

### BACKGROUND OF THE INVENTION

### Field of the Invention

The present invention relates to a coordinate transformation technique of synchronizing an ultrasound image with real space coordinates.

### Description of the Related Art

A surgical navigation system is a system for supporting surgery by displaying a position of a surgical instrument during surgery in real time on a medical image such as a computed tomography (CT) image or a magnetic resonance imaging (MRI) image and providing information on a positional relationship between a patient and the surgical instrument during surgery. In the surgery navigation, the CT image or the MRI image is excellent in terms of spatial resolution and contrast, but is poor in real-time property, and accuracy of the navigation decreases due to an influence of movement and deformation of an organ.

In order to solve this problem, there is a method for performing navigation while supplementing the real-time property by synchronously displaying the CT image or the MRI image and an ultrasound image. In order to implement the method, it is necessary to perform a registration operation of matching the medical image used for navigation with a position of the patient in the real space and an ultrasound probe registration operation of registering a position and an angle of a scanning surface of an ultrasound beam. In general, the registration operation and the ultrasound probe registration operation are performed by a surgeon before the surgery. An order of performing the registration operation and the ultrasound probe registration operation is not limited, and either the registration operation or the ultrasound probe registration operation may be performed first.

As a registration method, there are established a method for associating a position of a marker or the like in the real space with a position of a marker or the like on a medical image by indicating three or more points of anatomical landmarks such as a nose root and the outer corner of an eye and positions of imaging markers attached to a patient, and a method for associating surface information on the patient acquired using laser or the like with surface information on a three-dimensional image reconstructed from the medical image (Atsuro Koga, “Surgery Navigation System: Stealth Station”, Journal of the Kinki Subcommittee of Japanese Society of Radiological Technology, Vol. 10, No. 1 (Non-Patent Literature 1)).

Meanwhile, various methods are proposed as an ultrasound probe registration method. For example, JP-A-10-151131 discloses a method for registering a position and an angle of a scanning surface of an ultrasound beam in order to synchronize the scanning surface of the ultrasound beam from an ultrasound image to a CT image or an MRI image.

Further, https://www.youtube.com/watch?v=RAHgsUVm4d4 (Non-Patent Literature 2) discloses a method in which an ultrasound probe is fixed to an ultrasound probe registration tool to which a position detection marker is attached, and a position and an angle of a scanning surface of an ultrasound beam are registered based on the type of the ultrasound probe (Non-Patent Literature 2).

### SUMMARY OF THE INVENTION

The method of JP-A-10-151131 requires a complicated operation in order to register the position and angle of the scanning surface of the ultrasound beam, and has problems such as an increase in burden on the operator and an increase in operation time.

Meanwhile, in the method of Non-Patent Literature 2, the ultrasound probe is fixed to the ultrasound probe registration tool to which the position detection marker is attached and the position and the angle of the scanning surface of the ultrasound beam are registered based on the type of the ultrasound probe. Calibration of the ultrasound probe registration tool is necessary when registering ultrasound probes of different types. Even for ultrasound probes of the same type, the angle of the scanning surface is slightly different for each ultrasound probe. Since the registration tool in Non-Patent Literature 2 does not consider a difference in the angle of the scanning surface for each ultrasound probe, calibration of the ultrasound probe registration tool is necessary in order to accurately register the position and the angle of the scanning surface. Even for the same ultrasound probe, when the ultrasound probe is fixed to the ultrasound probe registration tool, registration accuracy may also be lowered if the ultrasound probe is not fixed at the same position with good reproducibility.

In this manner, although various methods are proposed for registering the position and the angle of the scanning surface of the ultrasound probe, there are still problems in terms of registration accuracy and operability.

An object of the invention is to register a position and an angle of a scanning surface of an ultrasound probe easily and accurately.

### Solution to Problem

In order to achieve the object, an ultrasound probe position registration method according to the invention includes arranging a phantom including two or more wires stretched in a non-parallel manner to a real space in which a position detection sensor is arranged, moving, in a parallel manner, an ultrasound probe to which a probe position detection marker is attached on the phantom while keeping an orientation of a main plane of the ultrasound probe constant, and acquiring two or more ultrasound images of the phantom while detecting a position of the probe position detection marker in the real space with the position detection sensor, and obtaining positions of cross-sectional images of the two or more wires included in each of the two or more ultrasound images, calculating a relation between the position of the probe position detection marker in the real space and an orientation and a position of the captured ultrasound images in the real space based on a relation between the obtained positions of the cross-sectional images, and registering the calculated relation as probe coordinate transformation information in a storage unit.

### Advantageous Effect

According to the invention, the position and the orientation of the scanning surface (ultrasound image) of the ultrasound beam can be calculated by a simple operation, the burden on the operator can be reduced, and the operability can be improved. Further, the position of the ultrasound probe can be accurately registered regardless of a type or an individual difference of the ultrasound probe using the ultrasound image of the phantom.

### BRIEF DESCRIPTION OF THE DRAWINGS

FIG. 1 illustrates a hardware configuration of an ultrasound imaging system according to an embodiment of the invention.

FIG. 2 is a perspective view of an ultrasound probe and a probe marker (probe position detection marker) according to the embodiment.

FIG. 3 illustrates a relation between an ultrasound probe position registration phantom according to the embodiment and a position and an angle of a scanning surface of an ultrasound beam.

FIG. 4 is a flowchart illustrating a procedure for registering a position of an ultrasound probe and performing surgery navigation using the ultrasound imaging system according to the embodiment.

FIG. 5 illustrates an example of a GUI for registering the position of the ultrasound probe according to the embodiment.

FIG. 6 illustrates an example of a surgery navigation screen of the ultrasound imaging system according to the embodiment, on which a CT image or an MRI image and an ultrasound image are synchronously displayed.

### DESCRIPTION OF THE PREFERRED EMBODIMENTS

Hereinafter, an embodiment of an ultrasound imaging system according to the invention will be described with reference to the drawings. In the following description and the drawings, components having the same functional configuration are designated by the same reference numerals to omit duplicate description.

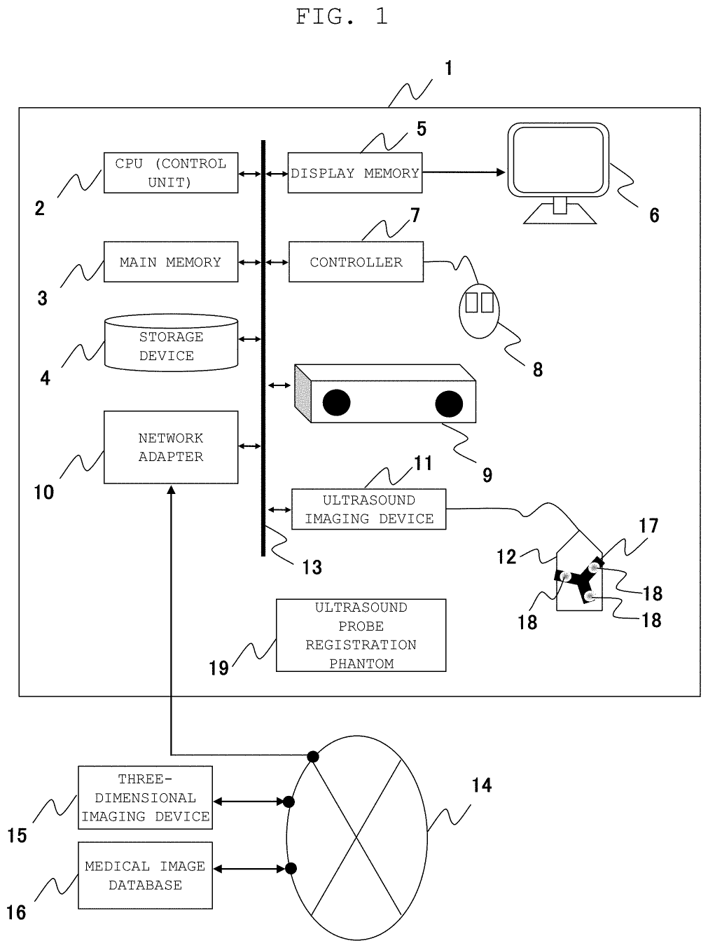

FIG. 1 illustrates a hardware configuration of an ultrasound imaging system 1. The ultrasound imaging system 1 includes a central processing unit (CPU) 2, a position detection sensor 9, an ultrasound imaging device 11, a network adapter 10, a main memory 3, a storage device 4, a display memory 5, a controller 7, and a display device 6, which are connected to each other by a system bus 13 so as to be capable of transmitting and receiving signals. Here, “capable of transmitting and receiving signals” means a state in which signals can be transmitted and received thereamong or from one to the other, regardless of whether they are connected electrically, optically, or wirelessly.

The ultrasound imaging device is connected with an ultrasound probe 12. As the ultrasound probe 12, for example, various probes 12 can be used, such as a sector probe, a linear probe, or a convex probe. The ultrasound probe 12 is equipped with the probe marker (probe position detection marker) 17.

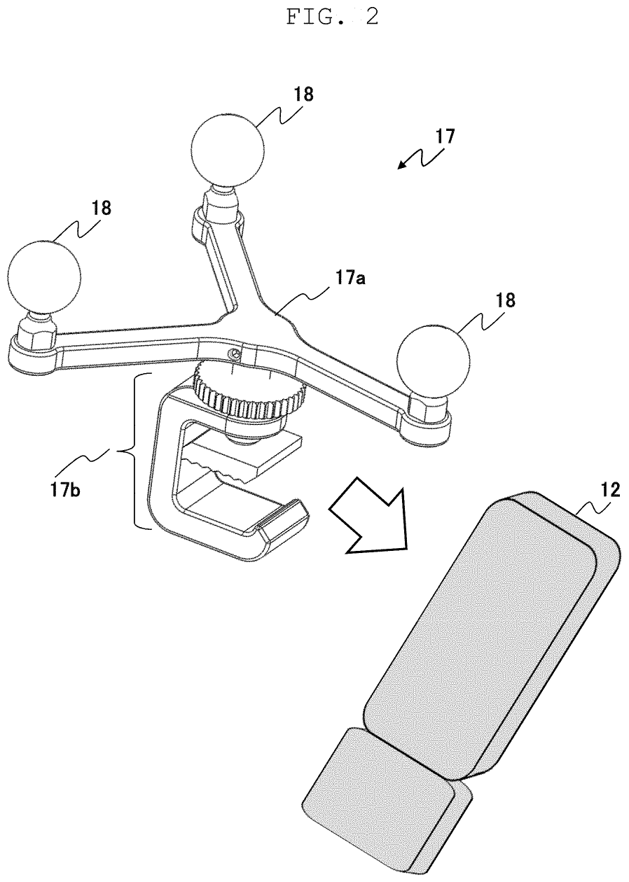

As illustrated in FIG. 2, the probe marker 17 includes a plurality of (here, three) balls 18, a frame 17a, and an attachment mechanism 17b. The frame 17a supports the plurality of balls 18 in a predetermined positional relation. The attachment mechanism 17b can fix the frame 17a to a predetermined position of the ultrasound probe 12 in a predetermined orientation. Each of the balls 18 is a reflector that reflects light such as visible light or infrared light, or a light source that emits light.

The position detection sensor 9 includes a pair of optical sensors that detect light reflected or emitted from the plurality of balls 18, and recognizes spatial coordinates of the plurality of balls 18. Accordingly, the position detection sensor 9 recognizes a position and an orientation of the ultrasound probe 12. As the position detection sensor 9, a magnetic field generation device may be used, and a magnetic detection sensor may be used instead of the probe marker 17.

The network adapter 10 is connected to a three-dimensional imaging device 15 such as a CT device or an MRI device and a medical image database 16 via a network 14 such as a local area network (LAN), a telephone line, or the internet so as to be capable of transmitting and receiving signals.

The storage device 4 stores a three-dimensional image captured by the three-dimensional imaging device 15 and a three-dimensional medical image read from the medical image database 16. The storage device 4 stores in advance a program executed by the CPU 2 and data necessary for executing the program. The storage device 4 is, specifically, a hard disk or the like, and may also be a device that exchanges data with a portable recording medium such as a flexible disk, a light (magnetic) disk, a ZIP memory, or a USB memory.

The CPU 2 implements a function as a control unit by software. The control unit controls an operation of each component by loading the program stored in advance in the storage device 4 and data necessary for program execution into the main memory 3 and executing the program (hereinafter, the CPU 2 is also referred to as the control unit 2). Functions of the control unit 2 may be implemented by hardware. For example, a custom IC such as an application specific integrated circuit (ASIC) or a programmable IC such as a field-programmable gate array (FPGA) may be used instead of the CPU 2 to design a circuit for implementing functions of the respective units.

The main memory 3 stores the program to be executed by the CPU 2 and a progress of an arithmetic processing.

The display memory 5 temporarily stores display data to be displayed on the display device 6. The display device 6 is a liquid crystal display, a cathode ray tube (CRT), or the like.

A mouse 8 is connected to the controller 7. The mouse 8 may be another pointing device such as a track pad or a trackball. The controller 7 detects a state of the mouse 8, acquires a position of a mouse pointer on the display device 6, and outputs the acquired position information and the like to the CPU 2.

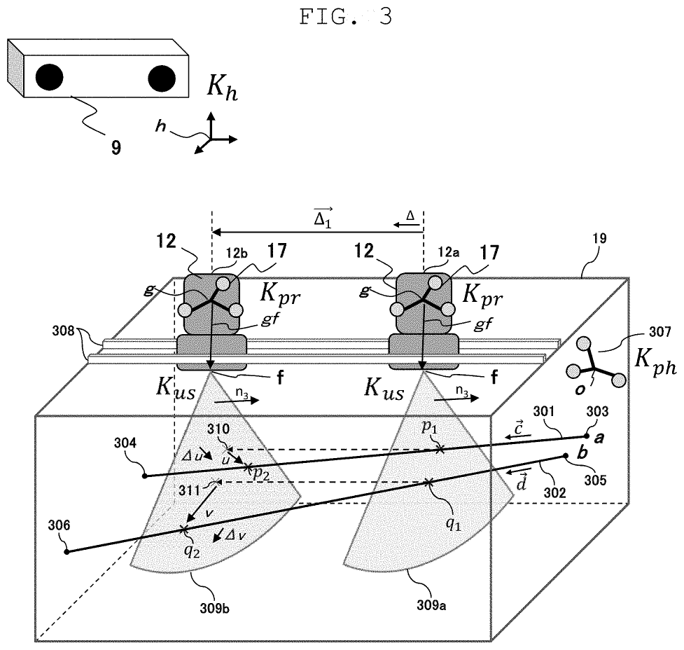

A structure of the phantom 19 is illustrated in FIG. 3. Two wires 301 and 302 are stretched in the phantom 19 in a non-parallel manner. A phantom marker 307 that can be detected by the position detection sensor 9 is fixed to the phantom 19, a point 303 and a point 304, which are fixed ends of the wire 301, and a point 305 and a point 306, which are fixed ends of the wire 302, are arranged at predetermined positions in a three-axis orthogonal coordinate system set in the phantom marker 307.

The phantom 19 is provided with a guide rail 308 that can slide (move in a parallel manner) the ultrasound probe in one direction while keeping an orientation (angle) of a main plane of the ultrasound probe constant. A position and a sliding direction of the guide rail 308 are fixed with respect to the phantom marker 307.

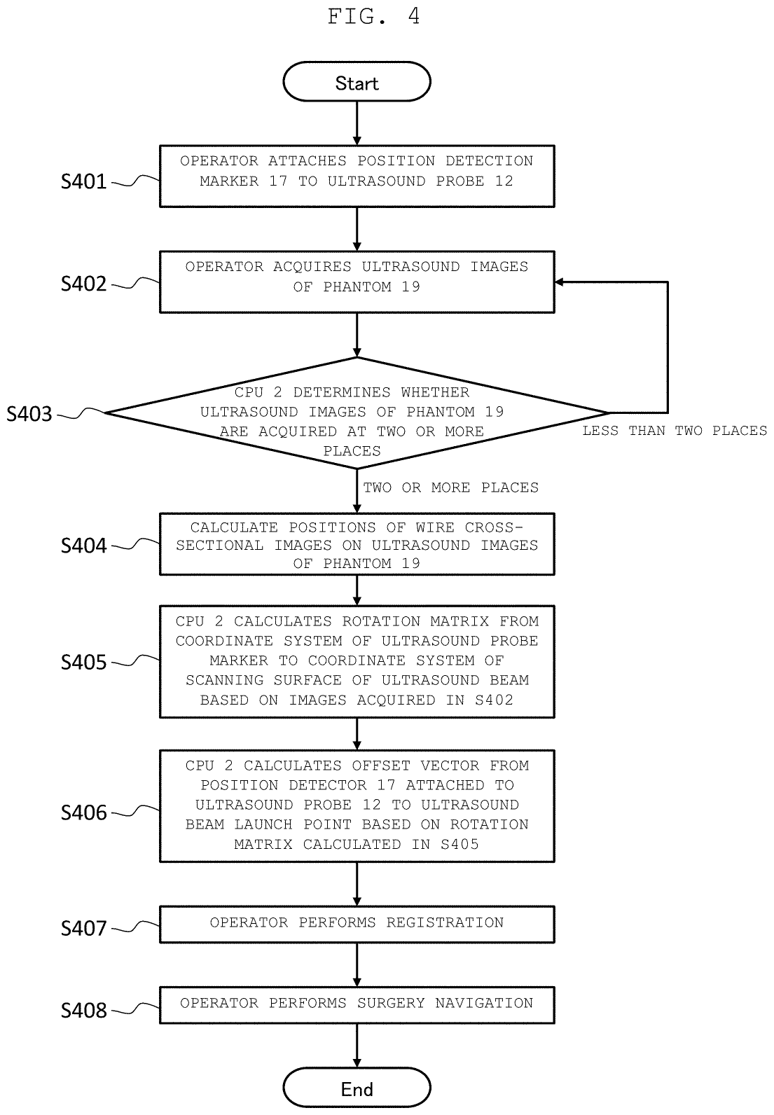

In the ultrasound imaging system according to the present embodiment, steps illustrated in FIG. 4 are executed in order.

The steps include a step of attaching the probe position detection marker to the ultrasound probe by an operator, a step of acquiring an ultrasound image of the ultrasound probe registration phantom (hereinafter, referred to as a phantom) 19 by the operator, a step of determining by the control unit 2 that the ultrasound image of the phantom 19 is acquired at two or more places, a step of detecting cross-sectional images of the wires 301 and 302 on the ultrasound image of the phantom 19 and registering wire positions, a step of calculating, by the control unit 2, a rotation matrix from a phantom coordinate system to a coordinate system of a scanning surface (a cross section of the captured ultrasound image) of an ultrasound beam based on the ultrasound image, a step of calculating, by the control unit 2, an offset vector from the probe position detection marker 17 to an ultrasound beam launch point based on the rotation matrix, a step of performing an operation for registering a subject and a medical image by the operator, and a step of, when the ultrasound image is acquired by moving the ultrasound probe 12 on the subject by the operator, generating a medical image corresponding to a position and an orientation of the ultrasound image from a position of the probe position detection marker 17 and displaying the medical image on the display device 6 by the control unit 2.

In this way, by calculating the position and the angle of the scanning surface of the ultrasound beam using the ultrasound image of the phantom, it is possible to provide a surgery navigation function in which a CT image or an MRI image and the ultrasound image are synchronously displayed can be provided.

FIG. 4 illustrates a basic flow of the invention. Hereinafter, each step illustrated in FIG. 4 will be described in detail.

### (Step S401)

In this step, the operator attaches the probe marker 17 to the ultrasound probe 12. The attachment mechanism 17b has a structure as illustrated in FIG. 2, for example, and the operator sandwiches the ultrasound probe 12 by screw tightening to fix the probe marker 17 to the ultrasound probe 12.

### (Step S402)

In this step, the ultrasound image of the phantom 19 is acquired. The phantom, which includes two or more wires stretched in a non-parallel manner, is disposed in the real space in which the position detection sensor 9 is disposed.

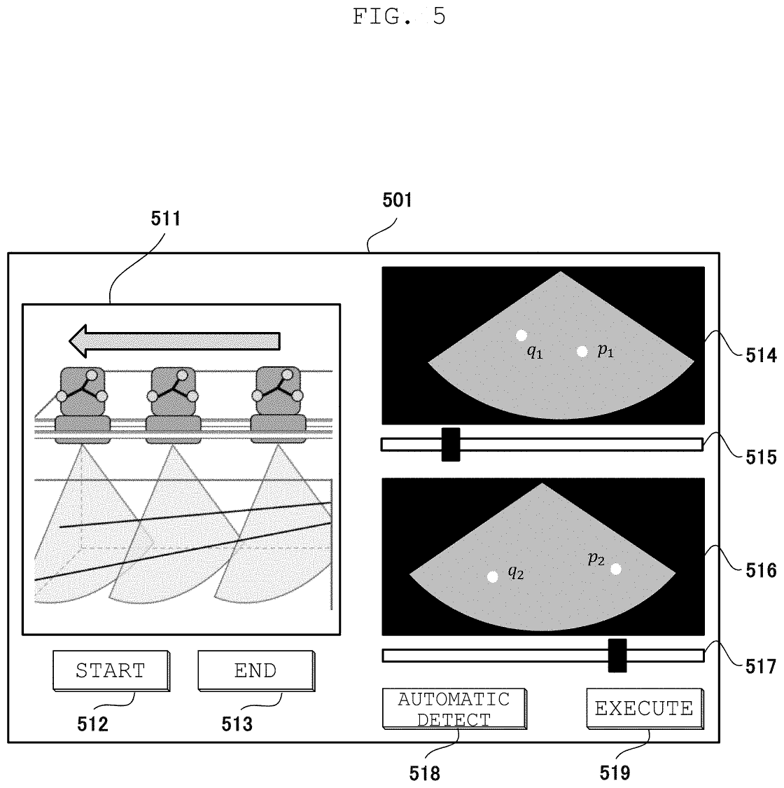

When acquiring the ultrasound image of the phantom 19, the control unit 2 displays GUI as illustrated in FIG. 5 on the display device 6. The operator attaches the ultrasound probe 12 to the guide rail 308, and fixes the ultrasound probe 12 so that an angle of a main plane of the ultrasound probe 12 with respect to the guide rail 308 (phantom 19) does not change. When the operator presses a GUI start button 512, the control unit 2 outputs a control signal to the ultrasound imaging device 11, and the ultrasound imaging device 11 starts to acquire the probe marker 17 and a corresponding ultrasound image. Alternatively, the control unit 2 may detect that the probe marker 17 is stationary within a predetermined range of the phantom marker 307, and instruct the start of acquiring the probe marker 17 and the corresponding ultrasound image.

The operator slides the ultrasound probe 12 along the guide rail 308 according to an operation guide animation 511 in the GUI illustrated in FIG. 5. The position detection sensor 9 detects a three-dimensional position of the probe marker 17. The ultrasound imaging device 11 captures an ultrasound image of the phantom 19 corresponding to the three-dimensional position. Accordingly, the ultrasound imaging device 11 acquires the ultrasound image at two or more places along the guide rail 308.

After sliding the ultrasound probe 12 to an end of the guide rail 308, the operator ends the ultrasound image acquisition operation of the phantom 19 by pressing an end button 513. Alternatively, the control unit 2 may detect that the ultrasound probe 12 has moved a distance corresponding to a length of the guide rail 308, and end the acquisition of the ultrasound image of the phantom 19.

The acquired ultrasound image is recorded in the main memory 3 together with the three-dimensional position of the probe marker 17 at the time of acquisition.

In the above description, a method in which the operator manually slides the ultrasound probe 12 along the guide rail 308 has been described. Alternatively, the ultrasound probe 12 may be slid along the guide rail 308 by being driven by a motor or the like. In this case, start and end timings of the ultrasound image acquisition can be controlled by being synchronized with drive start and end timings of the motor, and the operation by the start button 512 and the end button 513 is not necessary.

### (Step S403)

The control unit 2 refers to the ultrasound image recorded in the main memory 3, and checks whether the ultrasound image is acquired at two or more places. When the number of places at which the ultrasound image is acquired is less than two, a message indicating that the ultrasound image needs to be acquired again is displayed on the display device 6. When the operator confirms the message, the operation of S402 is performed again. When the ultrasound image is acquired at two or more places, S404 is performed.

### (Step S404)

The control unit 2 reads out two ultrasound images of the ultrasound image of the phantom 19 acquired at two or more places in S402 from the main memory 3 and displays the two ultrasound images in ultrasound image display regions 514 and 516.

The operator can select the position of the probe marker 17 at the time of acquiring the ultrasound image by operating sliders 515 and 517 displayed on the screen with the mouse. The control unit 2 reads out the ultrasound images from the main memory and displays the read ultrasound images in the ultrasound image display regions 514 and 516. Each of the ultrasound images constitutes a set with the position of the probe marker 17 selected by the operator.

When the operator presses an automatic detection button 518, the control unit 2 detects the cross-sectional images of the wires 301 and 302 on the ultrasound images displayed in the ultrasound image display regions 514 and 516 by Hough transform or the like, and registers wire positions p₁, p₂, q₁, and q₂. Alternatively, the operator may also register the wire positions by visually recognizing the wires on the ultrasound images and selecting the wire positions on the ultrasound image display regions 514 and 516.

### (Step S405)

When the operator presses an execution button 519, the control unit 2 calculates the position and the angle of the scanning surface (ultrasound image) of the ultrasound beam.

Hereinafter, an algorithm for calculating the position and the angle of the scanning surface of the ultrasound beam will be described with reference to FIG. 4. It is assumed that ultrasound images 309a and 309b are equivalent to the ultrasound images displayed in the ultrasound image display regions 514 and 516, respectively.

Assuming that the positions of the wire 301 in the ultrasound images 309a and 309b are p₁ and p₂, the positions of the wire 302 in the ultrasound images 309a and 309b are q₁ and q₂, respectively, a coordinate system set for the phantom marker 307 is Kₚₕ, an origin is o, the point 303 is a, the point 305 is b, a unit vector along the wire 301 is c, and a unit vector along the wire 302 is d, vectors opᵢ and oqᵢ from the origin o to pᵢ and qᵢ (i=1, 2) in the coordinate system Kₚₕ are expressed as follows using the coefficients s and t. In the following description, an arrow in the expression represents a vector.

\vec{opᵢ}=\vec{oa}+sᵢ\vec{c} (1)

\vec{oqᵢ}=\vec{ob}+tᵢ\vec{d} (2)

When a movement vector of the ultrasound probe 12 from 12a to 12b is set as a vector Δ₁, the following expressions are established using coefficients α and β.

s₂−s₁=α|\vec{Δ₁}| (3)

t₂−t₁=β|\vec{Δ₁}| (4)

Here, assuming that a point 310 is a point obtained by moving p₁ by the vector Δ₁ and a point 311 is a point obtained by moving q₁ by the vector Δ₁, the points 310 and 311 are points on the plane of 309b. A vector u from the point 310 to the point p₂ is expressed as follows.

\vec{u}=\vec{op₂}−(\vec{op₁}+\vec{Δ₁})=(α\vec{c}−\vec{Δ})|\vec{Δ₁}| (5)

Similarly, a vector v from the point 311 to the point q₂ is expressed as follows.

\vec{v}=(β\vec{d}−\vec{Δ})|\vec{Δ₁}| (6)

A vector Δu corresponding to the vector u when the ultrasound probe 12 is moved by the unit vector Δ in a sliding direction of the guide rail 308 is expressed as follows.

$$Δ \vec{u} = \frac{\vec{u}}{\vert \vec{{Δ}_{1}} \vert} = α \vec{c} - \vec{Δ} \qquad ( 7 )$$

Similarly, a vector Δv is expressed as follows.

$$Δ \vec{v} = \frac{\vec{v}}{\vert \vec{{Δ}_{1}} \vert} = β \vec{d} - \vec{Δ} \qquad ( 8 )$$

Here, by calculating |Δu|², the value of a can be calculated as follows.

|Δ\vec{u}|²=α²−2(\vec{c}·\vec{Δ})α+1⇔α=(\vec{c}·\vec{Δ})±\sqrt{(\vec{c}·\vec{Δ})²+|Δ\vec{u}|²−1} (9)

Here, by calculating |Δv|², the value of β can be calculated as follows.

β=(\vec{d}·\vec{Δ})±\sqrt{(\vec{d}·\vec{Δ})²+|Δ\vec{v}|²−1} (10)

Here, since the vector Δu and the vector Δv are present on the scanning surface of the ultrasound beam, a unit normal vector n₃ with respect to the scanning surface of the ultrasound beam can be calculated as follows.

$$\vec{{n}_{3}} = \frac{Δ \vec{u} \times Δ \vec{v}}{\vert Δ \vec{u} \times Δ \vec{v} \vert} \qquad ( 11 )$$

A rotation matrix R_{us←ph} from the coordinate system Kₚₕ set in the phantom marker 307 to a coordinate system Kᵤₛ of the scanning surface of the ultrasound beam can be calculated as follows. However, in expression 12, Δuₓ and Δu_{y} are an x component and a y component of Δu in the coordinate system Kᵤₛ, and Δvₓ and Δv_{y} are an x component and a y component of Δv in the coordinate system Kᵤₛ.

$${R}_{us ← ph} = ( \begin{matrix}{Δ u}_{x} & {Δ v}_{x} & 0 \\ {Δ u}_{y} & {Δ v}_{y} & 0 \\ 0 & 0 & 1\end{matrix} ) {( \begin{matrix}Δ \vec{u} & Δ \vec{v} & \vec{{n}_{3}}\end{matrix} )}^{- 1} \qquad ( 12 )$$

When a coordinate system set for the probe marker 17 attached to the ultrasound probe is set as Kₚᵣ and an origin is set as g, a coordinate system in the position detection sensor 9 is set as Kₕ and an origin is set as h, a transformation matrix R_{us←pr} from the coordinate system Kₚᵣ set for the probe marker 17 to the coordinate system Kᵤₛ of the scanning surface of the ultrasound beam is expressed as follows.

R_{us←pr}=R_{us←ph}R_{h←ph}⁻¹R_{h←pr} (13)

In expression 13, R_{h←ph} and R_{h←pr} are coordinate transformation matrices obtained by detecting the phantom marker 307 and the probe marker 17 by the position detection sensor. The position detection sensor detects three-dimensional positions of three or more balls attached to each position detection marker, recognizes a coordinate system of each position detection marker set with reference to arrangements of each ball, and calculates the coordinate transformation matrix from the coordinate system of each position detection marker to a sensor coordinate system.

### (Step S406)

When the launch point of the ultrasound beam is set as f, a vector op₁(Kₚₕ) is expressed as follows.

$$\vec{{op}_{1}} ( {K}_{ph} ) = \vec{og} ( {K}_{ph} ) + \vec{gf} ( {K}_{ph} ) + \vec{{fp}_{1}} ( {K}_{ph} ) = {R}_{h ← ph}^{- 1} ( \vec{hg} ( {K}_{h} ) - \vec{ho} ( {K}_{h} ) ) + {R}_{h ← ph}^{- 1} {R}_{h ← pr} gf ( {K}_{pr} ) + {R}_{ph ← us} \vec{{fp}_{1}} ( {K}_{us} ) \qquad ( 14 )$$

From expression 14, an offset vector gf(Kₚᵣ) from the probe marker 17 attached to the ultrasound probe 12 to the ultrasound beam launch point can be calculated.

The coordinate system set for the probe marker 17 attached to the ultrasound probe calculated in step S405 is registered as the rotation matrix R_{us←pr} from Kₚᵣ to the coordinate system Kᵤₛ of the scanning surface of the ultrasound beam, and the offset vector gf (Kₚᵣ) from the origin g of the probe marker 17 calculated in step S406 to the ultrasound beam launch point f is registered as the coordinate transformation information on the ultrasound probe 12.

The display device 6 displays that the registration of the ultrasound probe 12 is completed. When another ultrasound probe is desired to be registered, a plurality of ultrasound probes can be registered by repeating the operations of steps S401 to S406.

### (Step S407)

The operator performs a registration operation to match a position of a medical image used for navigation with a position of the subject (hereinafter, also referred to as a patient) in the real space. The registration is performed by a method (point registration) for associating a marker position in the real space with a marker position on the medical image by pointing three or more points of anatomical landmarks such as a nose root and the outer corner of an eye of the patient and positions of imaging markers attached to the patient, or a method (surface registration) for associating surface information on the patient acquired using laser or the like with surface information on the three-dimensional image reconstructed from the medical image (Non-Patent Literature 1). The registration operation may be performed before the ultrasound probe registration operation (before step S401).

### (Step S408)

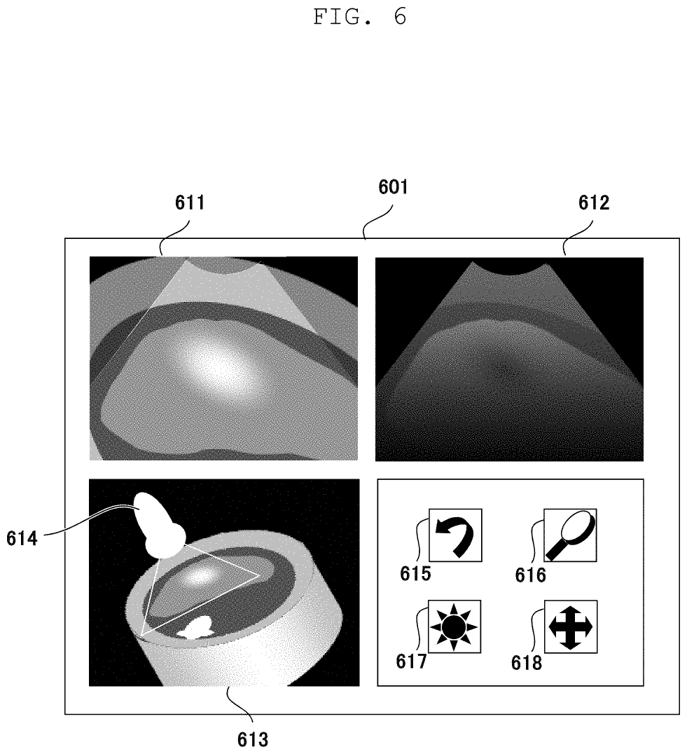

FIG. 6 illustrates a basic embodiment of the GUI at the time of performing the surgery navigation in which the CT image or the MRI image and the ultrasound image are synchronously displayed. When the ultrasound probe registered in step S406 is applied to a diseased part, an ultrasound image is displayed in an ultrasound image display region 612.

The CT image or the MRI image is associated with the patient position in the real space by the registration operation performed in step S407, and positions in the ultrasound image is associated with positions in the real space using the rotation matrix R_{us←ph} and the offset vector gf for the position information on the probe marker 17 attached to the ultrasound probe 12 detected by the position detection sensor 9. Accordingly, the medical image such as the CT image or the MRI image corresponding to a position where the ultrasound image is depicted is synchronously displayed in a navigation image display region 611.

According to the present embodiment, the system can automatically calculate the position and the angle of the scanning surface of the ultrasound beam by a simple operation of simply acquiring the ultrasound image of the phantom, the burden on the operator can be reduced, and the operability can be improved. Further, the ultrasound probe 12 can be accurately registered regardless of the type or the individual difference of the ultrasound probe 12 by calculating the position and the angle of the scanning surface of the ultrasound beam based on the ultrasound image of the phantom.

The registration of the coordinate transformation information on the ultrasound probe 12 in steps S401 to S406 described above is preferably performed by disposing the ultrasound imaging system 1 including the position detection sensor 9, the phantom 19, and the ultrasound imaging device in a room where the patient (subject) on which the surgical navigation is performed is arranged, and imaging the ultrasound image of the phantom 19 by the ultrasound probe 12 provided with the marker 17, and performing the surgery navigation using the same position detection sensor 9. Alternatively, the present embodiment is not limited thereto. The surgery navigation may be performed using another position detection sensor by disposing the ultrasound imaging system 1 including the position detection sensor 9, the phantom 19, and the ultrasound imaging device in a room separate from the room where the surgical navigation is performed, registering the coordinate transformation information on the ultrasound probe 12 by steps S401 to S406, and then moving only the ultrasound probe 12 provided with the marker 17 to the room where the patient (subject) is arranged and the other position detection sensor is disposed. In this case, although errors due to different position detection sensors may occur, the errors can be reduced by calibrating the position detection sensors.

What is claimed is:

1. An ultrasound probe position registration method comprising:
a first step of disposing a phantom including two or more wires stretched in a non-parallel manner in a real space in which a position detection sensor is disposed, moving an ultrasound probe, to which a probe position detection marker is attached, on the phantom in a parallel manner while keeping an orientation of a main plane of the ultrasound probe constant, and acquiring two or more ultrasound images of the phantom while detecting a position of the probe position detection marker in the real space with the position detection sensor; and
a second step of obtaining positions of cross-sectional images of the two or more wires included in each of the two or more ultrasound images, calculating a relation between the position of the probe position detection marker in the real space and orientations and positions of the captured ultrasound images in the real space based on a relation between the obtained positions of the cross-sectional images of the two or more wires, and registering the calculated relation as probe coordinate transformation information in a storage unit.

2. The ultrasound probe position registration method according to claim 1, wherein
the phantom is equipped with a phantom position detection marker and the two wires are stretched in a predetermined positional relation with the phantom position detection marker,
in the first step, a position of the phantom position detection marker in the real space is further detected by the position detection sensor, and
in the second step, as a relation between the position of the probe position detection marker in the real space and the orientations and the positions of the captured ultrasound images in the real space, a rotation matrix R_{us←pr} for transforming a coordinate system of the phantom position detection marker into a coordinate system of a scanning surface of an ultrasound beam of the ultrasound probe and an offset vector gf (Kₚᵣ) from an origin of the probe position detection marker to a launch point of the ultrasound beam of the ultrasound probe are calculated.

3. An ultrasound imaging system comprising:
a position detection sensor;
a phantom including two or more wires stretched in a non-parallel manner;
an ultrasound probe to which a probe position detection marker is attached;
an ultrasound imaging device that transmits and receives ultrasound waves by the ultrasound probe and capture an ultrasound image;
a storage unit; and
a control unit, wherein
the control unit is configured to
move the ultrasound probe in a parallel manner on the phantom disposed in the real space while keeping an orientation of a main plane of the ultrasound probe constant and receive two or more ultrasound images of the phantom captured by the ultrasound imaging device while detecting a position of the probe position detection marker in the real space with the position detection sensor,
calculate positions of cross-sectional images of the two or more wires included in each of the two or more ultrasound images,
calculate a relation between the position of the probe position detection marker in the real space and orientations and positions of the captured ultrasound images in the real space based on a relation between the positions of the cross-sectional images of the two or more wires, and
register the calculated relation as probe coordinate transformation information in the storage unit.

4. The ultrasound imaging system according to claim 3, wherein
the control unit is further configured to
receive an ultrasound image obtained by imaging a subject by the ultrasound imaging device and a position of the probe position detection marker detected by the position detection sensor at the time of imaging,
calculate an orientation and a position of the ultrasound image of the subject using the probe coordinate transformation information registered in the storage unit and the position of the probe position detection marker, and
generate a two-dimensional medical image corresponding to the orientation and the position of the ultrasound image of the subject from three-dimensional medical image data captured in advance for the subject, and display the two-dimensional medical image together with the ultrasound image on a connected display device.

5. The ultrasound imaging system according to claim 3, wherein
the phantom is equipped with a phantom position detection marker and the two wires are stretched in a predetermined positional relation with the phantom position detection marker,
the control unit further detects a position of the phantom position detection marker in the real space by the position detection sensor, and
as a relation between the position of the probe position detection marker in the real space and the orientation and the position of the captured ultrasound image in the real space, the control unit calculates a rotation matrix R_{us←pr} for transforming a coordinate system of the phantom position detection marker into a coordinate system of a scanning surface of an ultrasound beam of the ultrasound probe and an offset vector gf(Kₚᵣ) from an origin of the probe position detection marker to a launch point of the ultrasound beam of the ultrasound probe.

6. The ultrasound imaging system according to claim 3, wherein
the phantom includes a guide rail for moving the ultrasound probe in a parallel manner while keeping the orientation of the main plane of the ultrasound probe constant.

7. An ultrasound probe position registration system comprising:
a position detection sensor;
a phantom disposed in a real space in which the position detection sensor is disposed, the phantom including two or more wires stretched in a non-parallel manner;
a probe position detection marker attached to an ultrasound probe of an ultrasound imaging device;
a storage unit; and
a control unit, wherein
the control unit is configured to
move the ultrasound probe in a parallel manner on the phantom disposed in the real space while keeping an orientation of a main plane of the ultrasound probe constant and receive two or more ultrasound images of the phantom captured by the ultrasound imaging device while detecting a position of the probe position detection marker in the real space with the position detection sensor,
calculate positions of cross-sectional images of the two or more wires included in each of the two or more ultrasound images,
calculate a relation between the position of the probe position detection marker in the real space and orientations and positions of the captured ultrasound images in the real space based on a relation between the positions of the cross-sectional images of the two or more wires, and
register the calculated relation as probe coordinate transformation information in the storage unit.
